(19) United States
(12) Patent Application Publication
(10) Pub. No.: US 2005/0048628 A1
(43) Pub. Date: Mar. 3, 2005

(54) Immunological binding molecules inhibiting the syncytial fusion of trophoblast

(76) Inventors: Hans-Georg Frank, Kerkrade (NL); Peter Kaufmann, Vaals (NL); Ulrike Schmitz, Aachen (NL)

Correspondence Address: JACOBSON HOLMAN PLLC, 400 SEVENTH STREET N.W., SUITE 600, WASHINGTON, DC, 20004, US

(21) Appl. No.: 10/482,394

(22) PCT Filed: Jul. 3, 2002
(86) PCT No.: PCT/EP02/07359

(30) Foreign Application Priority Data
Jul. 4, 2001 (EP) 01116199.9

(51) Int. Cl. C07H 21/04; C12P 21/04; C07K 14/745

(52) U.S. Cl. 435/69.6; 435/320.1; 435/325; 530/383; 536/23.5

(57) ABSTRACT

A protein which is homologous with chicken VL or VH to at least 80% and inhibits syncytial fusion, as well as fragments having at least 12 continuous amino acids from the sequence of chicken VL or VH.

The present invention relates to proteins which inhibit the syncytial fusion of human trophoblast cells, medicaments containing such proteins, and the use thereof for contraception in mammals.

The inhibition of syncytial fusion is a principle of action which can be used for contraception. In this document, the term “contraception” is defined under clinical-practical aspects in such a way that a monthly menstruation occurs and no pregnancy begins despite of sexual intercourse. This general definition also applies to most of the contraceptives which are clinically usual today. Contraception in humans and other mammals can be achieved by applying any of a number of principles or substances. In the known methods, the fusion of oocytes and spermatozoa is prevented by mechanical or chemical means. The spermatozoon can be inactivated or prevented from undergoing fertilization, for example, by using condoms, intra-uterine implants or spermicidal substances. Further, it is possible to hinder the maturation of the oocyte in the maternal ovary, which is possible by hormonal methods. Such methods are known to the skilled person.

A large group of the hormonal contraceptives in use today (“the pill”) affect the mucosa of the uterus (the endometrium) in such a way that nidation of the blastocyst is no longer possible since the endometrium is no longer receptive. These endocrine methods of contraception include the problem that an intake of the hormones precise in time and dose must be ensured in order to achieve the desired effect. Since hormones themselves have only a short half-life, on the order of hours to days at longest, a successful contraception with this method is only possible with daily intakes or by administering depot preparations. All these solutions include the problem that they interfere with or prevent the cyclic endocrine homeostasis of the female organism. Since the endometrium is a component of the maternal organism, the maternal organism is also the preferred place of the occurrence of undesirable side-effects.

Therefore, methods and substances are desirable which:
a) have their site of action on the implanted blastocyst and thus can additionally minimize the side effects in the maternal organism;
b) do not interfere with the endocrine autoregulation of the female organism.

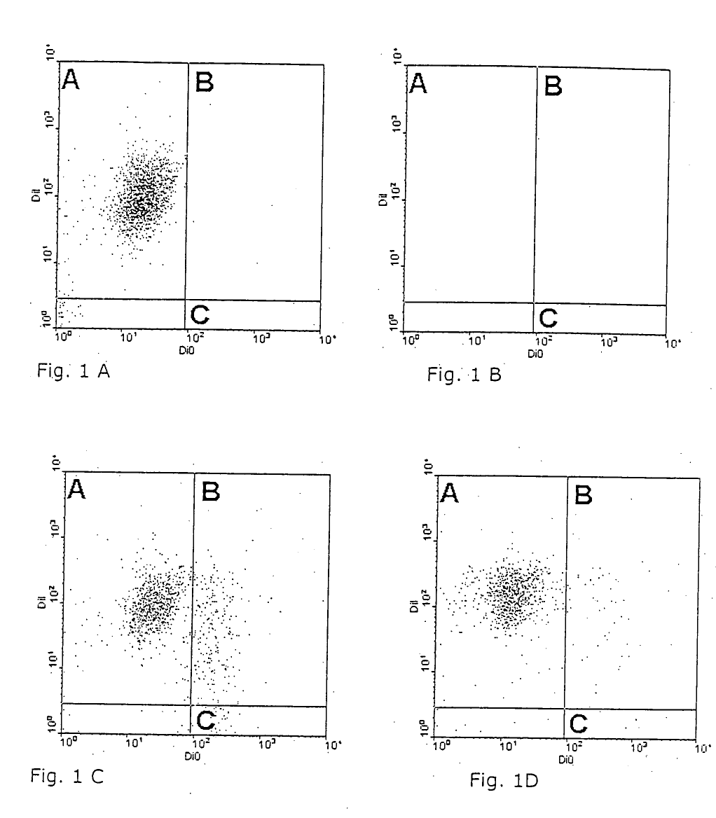

FIG. 1 shows an example of a flow-cytometric analysis within the scope of a fusion assay. In quadrant A, the fluorescence of DiI (alone) is represented, while in quadrant C, the fluorescence of DiO (alone) is represented. In quadrant B, cells become visible which exhibit a double fluorescence caused by fusion.

FIGS. 1A, B show controls in which the cells were stained with only one fluorescent dye each and incubated under the conditions of the assay. In FIG. 1A, staining was performed only with DiI, and in FIG. 1B only with DiO. The measured values for DiI are zero in the DiO-specific measuring channel; therefore, the points therein coincide with the X axis.

FIGS. 1C, D show the fusion assays of the clone PHSK04. FIG. 1C shows the rate of fusion of the cells under control conditions (in the presence of a non-specific protein, expressed in the same expression system and purified), and FIG. 1D shows the fusion rate in the presence of 100 nM PHSK04 protein.

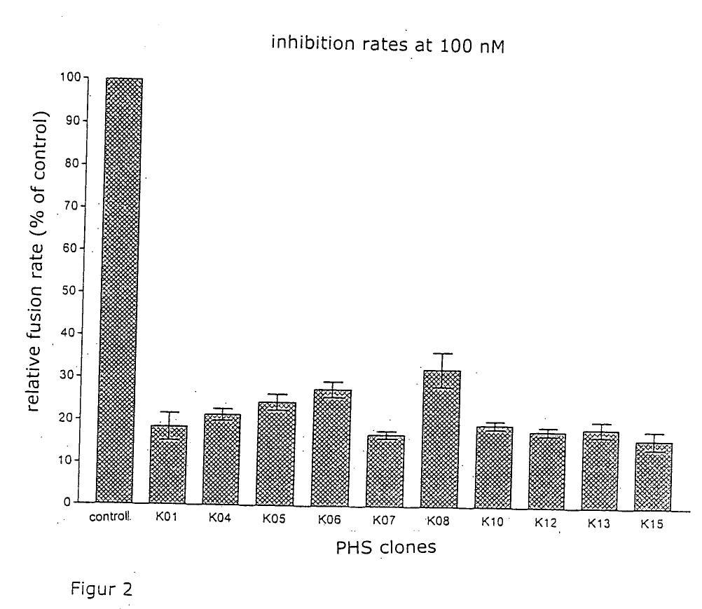

FIG. 2 shows the relative inhibition rates of the exemplary clones in the fusion assay as compared with a control (calmodulin, expressed in the same expression system and purified). Mean values from three determinations are represented.

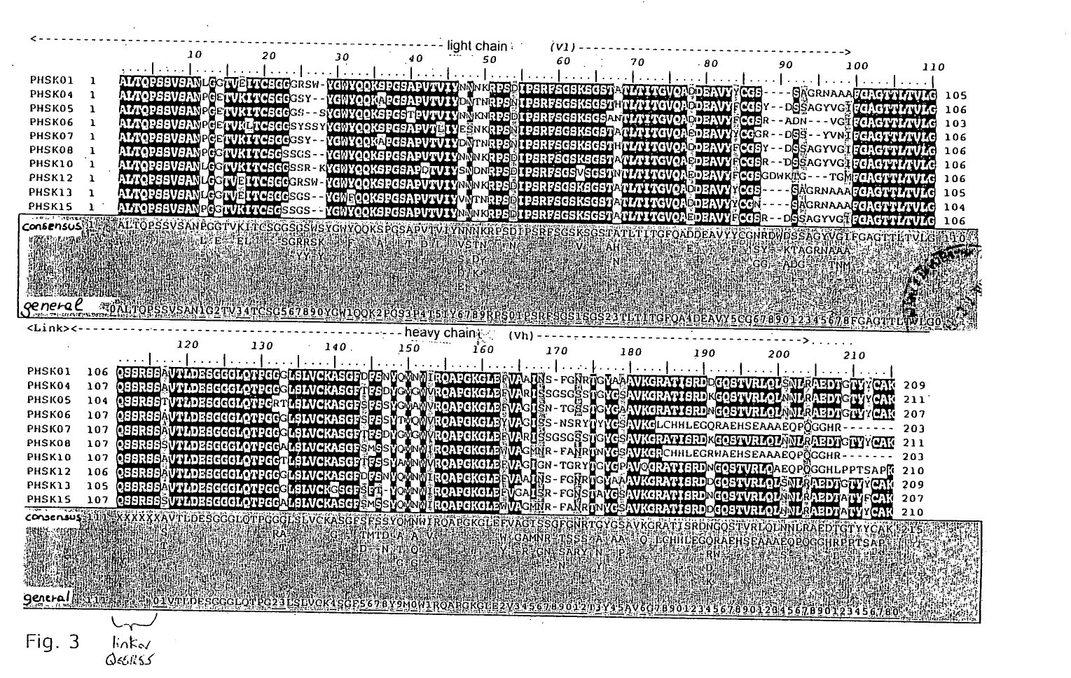

FIG. 3 shows the consensus sequence for the proteins preferred according to the invention with the SEQ ID Nos. 1 to 10. In the exemplary embodiment represented here, the linker is the sequence QSSRSS. The consensus sequences for VL and VH are represented in the one-letter code below the individual sequences of the clones with gray-shading. The consensus sequence itself is to be read horizontally while preferred alternative amino acids are shown vertically for each position in the one-letter code. A hyphen in this place means that the corresponding position may also be lacking in comparison with the consensus sequence. An X means that no portion of the consensus sequences for VL or VH is present in this place.

In the presence of PHSK04 protein, the fusion rate is decreased.

The object of the invention is achieved by a protein which is homologous with chicken VL or VH to at least 80% and inhibits syncytial fusion, as well as fragments having at least 12 continuous amino acids from the sequence of chicken VL or VH. In preferred embodiments, the protein according to the invention is homologous with chicken VL or VH to at least 85% or at least 90%. In preferred embodiments, the fragments according to the invention have at least 20 or 30 continuous amino acids from the sequence of chicken VL or VH.

Preferably, the protein VL according to the invention has the following amino acid sequence:

X⁰ A L T Q P S S V S A N X¹ G X² T V X³ X⁴ T C S G
X⁵ X⁶ X⁷ X⁸ X⁹ X¹⁰ Y G W X¹¹ Q Q K X¹² P G S X¹³ P
X¹⁴ T X¹⁵ I Y X¹⁶ X¹⁷ X¹⁸ X¹⁹ R P S X²⁰ I P S R F
S G S X²¹ S G S X²² X²³ T L T I T G F Q A X²⁴ D E
A V Y X²⁵ C G X²⁶ X²⁷ X²⁸ X²⁹ X³⁰ X³¹ X³² X³³ X34
X³⁵ X³⁶ X³⁷ X³⁸ F G A G T T L T V L G X³⁹

wherein
X⁰=an unsubstituted, mono- or disubstituted amino group or one or more amino acids;
X¹=P or L or a deletion;
X²=G or E or a deletion;
X³=K or E or a deletion;
X⁴=I or L or a deletion;
X⁵=G or S or a deletion;
X⁶=S or G or a deletion;
X⁷=G or R or Y or a deletion;
X⁸=S or R or Y or a deletion;
X⁹=W or S or a deletion;
X¹⁰=S or K or Y or a deletion;
X¹¹=Y or F or a deletion;
X¹²=S or A or a deletion;
X¹³=A or T or a deletion;
X¹⁴=V or D or a deletion;
X¹⁵=V or L or a deletion;
X¹⁶=N or V or S or D or E or a deletion;
X¹⁷=N or S or a deletion;
X¹⁸=N or T or D or K or a deletion;
X¹⁹=K or N or a deletion;
X²⁰=D or N or a deletion;
X²¹=K or V or a deletion;
X²²=T or A or a deletion;
X²³=A or H or N or a deletion;
X²⁴=D or E or a deletion;
X²⁵=Y or F or a deletion;
X²⁶=N or S or G or a deletion;
X²⁷=R or Y or G or a deletion;
X²⁸=D or a deletion;
X²⁹=W or a deletion;
X³⁰=D or K or A or a deletion;
X³¹=S or T or D or a deletion;
X³²=S or A or G or a deletion;
X³³=A or G or a deletion;
X³⁴=G or R or a deletion;
X³⁵=Y or N or a deletion;
X³⁶=V or A or T or a deletion;
X³⁷=G or A or N or a deletion;
X³⁸=I or A or M or a deletion; and
X³⁹=a carboxy group, a carboxy derivative or one or more amino acids;
and wherein mutual conservative exchanges of any amino acids are admissible as long as the inhibition of syncytial fusion is retained.

In another preferred embodiment, the protein according to the invention has the following sequence in VH:

X⁰ X¹ V T L D E S G G G L Q T P G X² X³ L S L V C
K X⁴ S G F X⁵ X⁶ X⁷ X⁸ Y X⁹ M X¹⁰ W X¹¹ R Q A P G
K G L E X¹² V X¹³ X¹⁴ X¹⁵ X¹⁶ X¹⁷ X¹⁸ X¹⁹ X²⁰ X²¹
X²² T X²³ Y X²⁴ X²⁵ A V X²⁶ G X²⁷ X²⁸ X²⁹ X³⁰ X³¹
X³² X³³ X³⁴ X³⁵ X³⁶ X³⁷ X³⁸ X³⁹ X⁴⁰ X⁴¹ X⁴² X⁴³
X⁴⁴ X⁴⁵ X⁴⁶ X⁴⁷ X⁴⁸ X⁴⁹ X⁵⁰ X⁵¹ X⁵² X⁵³ X⁵⁴ X⁵⁵
X⁵⁶ X⁵⁷ X⁵⁸ X⁵⁹

wherein
X⁰=an unsubstituted, mono- or disubstituted amino group or one or more amino acids;
X¹=A or S or T or a deletion;
X²=G or R or a deletion;
X³=G or A or T or a deletion;
X⁴=A or G or a deletion;
X⁵=S or T or D or a deletion;
X⁶=F or M or a deletion;
X⁷=S or T or a deletion;
X⁸=S or D or N or a deletion;
X⁹=G or Q or A or T or a deletion;
X¹⁰=N or A or Q or G or a deletion;
X¹¹=I or V or a deletion;
X¹²=F or W or Y or a deletion;
X¹³=A or G or a deletion;
X¹⁴=G or A or R or a deletion;
X¹⁵=I or M or a deletion;
X¹⁶=S or N or G or a deletion;
X¹⁷=S or R or N or a deletion;
X¹⁸=G or a deletion;
X¹⁹=F or T or S or N or a deletion;
X²⁰=G or S or A or a deletion;
X²¹=N or S or R or a deletion;
X²²=R or S or Y or a deletion;
X²³=G or A or N or Y or a deletion;
X²⁴=G or A or a deletion;
X²⁵=S or A or P or a deletion;
X²⁶=K or Q or a deletion;
X²⁷=R or L or a deletion;
X²⁸=A or C or a deletion;
X²⁹=T or H or a deletion;
X³⁰=I or H or a deletion;
X³¹=S or L or a deletion;
X³²=R or E or a deletion;
X³³=D or G or a deletion;
X³⁴=N or Q or R or D or K or a deletion;
X³⁵=G or R or W or a deletion;
X³⁶=Q or A or a deletion;
X³⁷=S or E or a deletion;
X³⁸=T or H or a deletion;
X³⁹=V or S or a deletion;
X⁴⁰=R or E or a deletion;
X⁴¹=L or A or a deletion;
X⁴²=Q or A or a deletion;
X⁴³=L or A or a deletion;
X⁴⁴=N or E or S or a deletion;
X⁴⁵=N or Q or a deletion;
X⁴⁶=L or P or a deletion;
X⁴⁷=R or Q or a deletion;
X⁴⁸=A or G or a deletion;
X⁴⁹=E or G or a deletion;
X⁵⁰=D or H or a deletion;
X⁵¹=T or R or L or a deletion;
X⁵²=G or P or a deletion;
X⁵³=T or P or a deletion;
X⁵⁴=Y or T or a deletion;
X⁵⁵=Y or S or a deletion;
X⁵⁶=C or A or a deletion;
X⁵⁷=A or P or a deletion;
X⁵⁸=K or a deletion;
X⁵⁹=a carboxy group, a carboxy derivative or one or more amino acids;
and wherein mutual conservative exchanges of any amino acids are admissible as long as the inhibition of syncytial fusion is retained.

According to the invention, “conservative exchanges” means, in particular, those which exchange amino acids within the groups of polar, non-polar, aromatic, charged amino acids. For example, glycine against alanine or valine etc., arginine or lysine against histidine etc., phenylalanine against tyrosine etc.

According to the invention, the proteins according to the invention can be interconnected by a linker. The linker is preferably a peptide chain of at least any five amino acids. Those amino acids are preferred which do not form any, or form only insignificant, interactions with the VL or VH chain. Instead of the peptide chain, the linker may also consist of organic compounds which are essentially formed from a chain of carbon atoms which may also comprise hetero atoms. In this case, the length of the linker is preferably at least nine Angströms.

The presence of D-amino acids in the sequence of the protein according to the invention can be of advantage because this may lead to a deceleration of degradation.

Especially preferred are the proteins according to the invention having the amino acid sequences SEQ ID Nos. 1 to 10.

These proteins are described by a consensus sequence having the sequence SEQ ID No. 11, to which the present invention also relates.

The invention also relates to nucleic acids, especially DNA or RNA, coding for a protein according to the invention, and a plasmid or vector containing such a nucleic acid.

The invention also relates to medicaments containing at least one of the proteins or nucleic acids according to the invention.

According to the invention, the use of at least one of the proteins or nucleic acids according to the invention for preparing a medicament for contraception in mammals is also claimed. The medicament according to the invention is typically administered in amounts of from 100 ng/kg to 1000 μg/kg of body weight. The route of administration is preferably parenteral, by infusion or by intramuscular or subcutaneous injection. Further possible dosage forms include a nasal spray, suppositories, vaginal application, e.g., via a tampon or other suitable application forms which are per se known to those skilled in the art of galenics or can be established in simple experiments.

A successful implantation of the blastocyst into the endometrium is not only dependent on the receptivity of the endometrium. Implantation is possible only if the cells in the outer layer of the blastocyst, the so-called trophoblast cells, fuse to form a syncytium. The formation of a syncytium is a process which occurs only in a few places in the biology of the human body. For example, this is known in the formation of skeletal muscle fibers and in the formation of osteoclasts.

In the syncytial fusion of myoblasts or in the formation of osteoclasts, molecules from the group of ADAM (a disintegrin and a metalloproteinase; for a survey, see Huppertz et al., 2001) are of particular importance while the trophoblast of humans uses a protein of an endogenous retrovirus, namely syncytin (Mi et al., 2000). Thus, there is a starting point for achieving a contraceptive effect by inhibiting the fusion of trophoblast cells. The process of trophoblast fusion is specific of trophoblasts, and therefore, its inhibition can be used for the selective trophoblast-specific contraception.

Functionally, the proteins according to the invention are characterized by their capability of inhibiting the syncytial fusion of trophoblast cells. They can be used for achieving a contraceptive effect in female mammals, especially rodents, pets, such as dogs and cats, and in humans. Therefore, the invention essentially comprises the following components: proteins which may preferably be in the form of immunological binding molecules which
a) can be classified as derivatives of the antibodies of a non-mammal, especially chicken, by a homology comparison; particularly, they are characterized materially by mentioning an amino acid consensus sequence;
b) are functionally characterized by the property of inhibiting the syncytial fusion of mammal trophoblast cells.
   Building Blocks of the Immunological Binding Molecules

In a preferred embodiment, the proteins according to the invention can be in the form of immunological binding molecules. In the Example described herein, the immunological binding molecules are recombinant molecules. The immunological binding molecules stated in the Examples are of the type “single chain variable fragment” antibody (scFv). Such an antibody molecule only consists of the variable regions of the light (VL) and heavy (VH) chains of an antibody molecule which are interconnected by a short sequence (linker) inserted by recombinant technology. Therefore, an scFv can be considered a minimalistic embodiment of an immunological binding molecule. The critical binding information is contained in the individual variable regions VL and VH, but not in the kind of their linking. Further embodiments of the present invention relate to the so-called Fab (antigen-binding fragment) antibody or other antibody molecules of any construction, optionally also non-recombinant natural antibody molecules, which contain at least one of the variable regions involved within their sequence. In the embodiment of scFv, which is also represented in the Examples, the following sequences of the modules involved are possible:
VL-linker-VH
VH-linker-VL

For other support constructions, it is also true that the order of VL and VH in the constructs is not predetermined, and that the construct itself does not critically contribute to the binding properties. “Support constructions” as used herein means those molecules onto which the scFv antibodies or parts of these antibodies can be mounted in order that they can find their binding partners in an optimum way.

Therefore, the present invention especially relates to all immunological binding molecules and their derivatives whose specifically binding regions have the consensus sequences stated below for VH and VL or are identical in sequence to at least 80% with the consensus sequence for VL or VH, respectively, and can inhibit the fusion of trophoblast cells. Also, it is not essential to the present invention whether so-called epitope tags (e.g., HIS tag for improved purification or myc tag for the use of secondary antibodies in immunological detection methods etc.) are introduced for technical reasons in the recombinant expression of the immunological binding molecules. These tags are of a purely technical nature and are not part of the sequences to be protected here.

Immunological binding molecules such as those described herein can be obtained from chickens by transferring the immune repertoire of chickens in a suitable form into molecular-biological libraries. Suitable techniques have been described, for example, in Barbas C F, Burton D R, Scott J K, Silverman G J; Phage Display: A laboratory manual, CSHL Press, New York. If such libraries are available, the molecules desired can be enriched by isolating, by means of known techniques (e.g., as described in Barbas et al., see above), those antibodies which bind to the surface of trophoblast cells which are ready for fusion. Subsequently, these antibodies are cloned by isolation on the DNA level and sequenced on the DNA level. Then, for checking their fusion-inhibiting effect, all antibodies must be tested and characterized in a fusion assay. The sequences of such fusion-inhibiting antibodies isolated from stochastic libraries are covered with a high probability by the consensus sequence stated here.

Alternatively, antibody DNA of a known sequence can be prepared by cloning chicken antibodies in a phage display by the above method and arbitrarily selecting and sequencing individual clones. The framework sequences of the chicken antibodies are highly homologous and deviate little from the VH and VL framework sequences herein. The binding specificity is ensured by the “complementarity-determining regions” (CDR) which are very different from one antibody to another. By selectively exchanging individual segments, primarily the CDR regions, using known molecular-biological techniques, antibodies of a desired sequence may also be established in this way, modifying the cloned chicken sequences.

### EXAMPLES

Antibody Molecules

The scFv antibodies set forth in the Example are contained in the vector pAC, encoded in DNA. This vector is an expression vector suitable for E. coli which consists of an f1 origin, an arabinose-controlled pBAD promoter and a gene which confers resistance to ampicillin. The DNA sequences coding the immunological binding molecules are inserted into the vector in such a way that their expression is controlled by the pBAD promoter. A signal peptide contained in the vector directs the expressed proteins into the periplasm of E. coli, and a 6×HIS tag is attached to the sequence to be expressed in order to enable purification by metal affinity chromatography. All clones are verified by sequencing prior to expression, so that it is established with no doubt that the DNA sequences employed have been correctly built into the vector.

The sequences can be synthesized as a protein in any expression system suitable for the expression of proteins, in both E. coli and Saccharomyces cerevisiae, for example, after appropriate recloning. The corresponding procedures are set forth in many text books of molecular biology and biotechnology, and all of them can be applied to the present sequences in principle. Such techniques are known to the skilled person. In the following, the expression via the vector pAC in E. coli (strain Top Ten, Invitrogen, Groningen, Netherlands) and the purification and characterization of the expressed proteins is described.

The pAC derivatives which contain the sequences to be expressed are first transformed into E. coli Top Ten by electroporation, and the transformation mixtures are plated onto agar plates (LB medium, 50 μg/ml ampicillin) and incubated over night at 30° C.

The following day, 5 ml each of LB medium (composition see Sambrook et al., 1989; containing 50 μg/ml of ampicillin) is inoculated with bacteria taken up from an individual colony. This culture is incubated over night at 37° C. on an incubation shaker at 300 revolutions per minute (rpm). The following day, the expression culture is inoculated from this over-night preculture at a dilution of 1:100 and incubated at 37° C. in a Fernbach flask at 300 rpm until an optical density (measured at 600 nm: OD600) of 0.5 has been reached. As a rule, this is the case after about 2.5 h. After the desired OD600 has been reached, the expression is induced by adding L-arabinose (up to a maximum concentration of 0.02%; the optimum concentration must be established individually for each clone in preliminary experiments). The expression is now performed at 26° C. and 300 rpm for 4-6 h. The growth behavior of the bacteria is documented by sampling and measuring OD600. Subsequently to this expression phase, the culture is centrifuged at 4° C. and 4000×g for 20 min, and the pellet is resuspended in 2 ml of TES buffer (0.2 M Tris-HCl, pH 8.0; 0.5 mM EDTA; 0.5 M sucrose). Now, 3 ml of ⅕ TES buffer (1 volume part of TES buffer plus 4 volumes of aqua bidestillata) is slowly added to this suspension, which is mixed and incubated on ice for 30 min. Thereafter, the sample is aliquoted into 1.5 ml reaction vessels and centrifuged at 4° C. and 10,000×g for 4° C. The supernatant then contains the periplasmic proteins and thus also the desired expression product.

For further processing, the supernatant is dialyzed over night at 4° C. against isotonic phosphate-buffered saline (pH 7.4, containing 10 mM imidazole). By means of commercially available kits for nickel affinity chromatography (e.g., Qiagen GmbH, Hilden, Germany), the desired protein can now be purified. The purification is controlled by gel electrophoresis, and the protein concentration is determined (Harlow and Lane, 1988). Molar concentrations were determined from the determination of concentration and the formula weight derived from the sequence.

The purified and quantitatively analyzed proteins are subsequently checked in a fusion assay under equimolar conditions for whether they can inhibit the fusion of human trophoblast cells.

In the following, the clones expressed and analyzed in the Example are listed, and the amino acid sequences translated from DNA sequences are represented in alignment.

Fusion Assay, Performance and Results

The proof of the fusion-inhibiting effect is furnished using the spontaneously fusing human trophoblast cell line AC-1M17. Cells of this cell line exhibit an initial fusion rate of 20-30% immediately after seeding into a new culture vessel.

Cells of this cell line are first proliferated to a sufficient number and then divided into two populations, which are stained with different commercially available fluorescent dyes (DiI and DiO, staining according to the instructions of the supplier Molecular Probes BV, Leiden, Netherlands).

After the cells have been thoroughly washed (5×10 minutes) to wash away any attached dye residues not integrated into the cell membrane, the two cell populations are mixed and commonly incubated at a low cell density for 24 hours under standard culturing conditions (5% carbon dioxide, 38° C., over 90% humidity) in Ham's F12 (10% fetal calf serum and antibiotics). After this culture phase, the cells are detached from the bottom of the culture flask by means of trypsin, suspended, and in the flow cytometer, it is determined how many of the cells present have adopted double fluorescence by fusion. FIG. 1 shows an example of such an evaluation of the fusion assay. FIG. 2 summarizes the inhibition rates of the fusion of these trophoblast cells as measured for the individual scFv clones stated in the Example. The Figure also shows the concentration dependence of the fusion-inhibiting effect in the various clones.

### List of References

Harlow E, Lane D (1988) Antibodies: A laboratory manual. Cold Spring Harbor Laboratory, Cold Spring Harbor, N.Y.
Huppertz B, Tews D S, Kaufmann P (2001) Apoptosis and syncytial fusion in human placental trophoblast and skeletal muscle. Int Rev Cytol 205:215-253
Mi S, Lee X, Li X, Veldman G M, Finnerty H, Racie L, LaVallie E, Tang X -Y, Edouard P, Howes S, Keith J C, McCoy J M (2000) Syncytin is a captive retroviral envelope protein involved in human placental morphogenesis. Nature 403:785-789
Sambrook J, Fritsch E F, Maniatis T (1989) Molecular cloning: a laboratory manual, 2nd ed. edn. Cold Spring Harbor Laboratory, Cold Spring Harbor, N.Y.

1. A protein which is homologous with chicken VL or VH to at least 80% and inhibits the syncytial fusion of mammal trophoblasts, wherein VL has the following sequence:
X⁰ A L T Q P S S V S A N X¹ G X² T V X³ X⁴ T C S G
X⁵ X⁶ X⁷ X⁸ X⁹ X¹⁰ Y G W X¹¹ Q Q K X¹² P G S X¹³ P
X¹⁴ T X¹⁵ I Y X¹⁶ X¹⁷ X¹⁸ X¹⁹ R P S X²⁰ I P S R F
S G S X²¹ S G S X²² X²³ T L T I T G F Q A X²⁴ D E
A V Y X²⁵ C G X²⁶ X²⁷ X²⁸ X²⁹ X³⁰ X³¹ X³² X³³ X³⁴
X³⁵ X³⁶ X³⁷ X³⁸ F G A G T T L T V L G X³⁹
wherein
X⁰=an unsubstituted, mono- or disubstituted amino group or one or more amino acids;
X¹=P or L;
X²=G or E;
X³=K or E;
X⁴=I or L;
X⁵=G or S;
X⁶=S or G;
X⁷=G or R or Y;
X⁸=S or R or Y or a deletion;
X⁹=W or S or a deletion;
X¹⁰=S or K or Y or a deletion;
X¹¹=Y or F;
X¹²=S or A or a deletion;
X¹³=A or T or a deletion;
X¹⁴=V or D;
X¹⁵=V or L;
X¹⁶=N or V or S or D or E;
X¹⁷=N or S;
X¹⁸=N or T or D or K;
X¹⁹=K or N;
X²⁰=D or N;
X²¹=K or V;
X²²=T or A;
X²³=A or H or N;
X²⁴=D or E;
X²⁵=Y or F;
X²⁶=N or S or G;
X²⁷=R or Y or G or a deletion;
X²⁸=D or a deletion;
X²⁹=W or a deletion;
X³⁰=D or K or A or a deletion;
X³¹=S or T or D;
X³²=S or A or G;
X³³=A or G or a deletion;
X³⁴=G or R or a deletion;
X³⁵=Y or N or a deletion;
X³⁶=V or A or T;
X³⁷=G or A or N;
X³⁸=I or A or M; and
X³⁹=a carboxy group, a carboxy derivative or one or more amino acids;
and wherein mutual conservative exchanges of any amino acids are admissible as long as the inhibition of the syncytial fusion of mammal trophoblasts is retained;
wherein VH has the following sequence:
X⁰ X¹ V T L D E S G G G L Q T P G X² X³ L S L V C
K X⁴ S G F X⁵ X⁶ X⁷ X⁸ Y X⁹ M X¹⁰ W X¹¹ R Q A P G
K G L E X¹² V X¹³ X¹⁴ X¹⁵ X¹⁶ X¹⁷ X¹⁸ X¹⁹ X²⁰ X²¹
X²² T X²³ Y X²⁴ X²⁵ A V X²⁶ G X²⁷ X²⁸ X²⁹ X³⁰ X³¹
X³² X³³ X³⁴ X³⁵ X³⁶ X³⁷ X³⁸ X³⁹ X⁴⁰ X⁴¹ X⁴² X⁴³
X⁴⁴ X⁴⁵ X⁴⁶ X⁴⁷ X⁴⁸ X⁴⁹ X⁵⁰ X⁵¹ X⁵² X⁵³ X⁵⁴ X⁵⁵
X⁵⁶ X⁵⁷ X⁵⁸ X⁵⁹
wherein
X⁰=an unsubstituted, mono- or disubstituted amino group or one or more amino acids;
X¹=A or S or T;
X²=G or R;
X³=G or A or T;
X⁴=A or G;
X⁵=S or T or D;
X⁶=F or M;
X⁷=S or T;
X⁸=S or D or N or a deletion;
X⁹=G or Q or A or T;
X¹⁰=N or A or Q or G;
X¹¹=I or V;
X¹²=F or W or Y;
X¹³=A or G;
X¹⁴=G or A or R;
X¹⁵=I or M;
X¹⁶=S or N or G;
X¹⁷=S or R or N;
X¹⁸=G or a deletion;
X¹⁹=F or T or S or N;
X²⁰=G or S or A;
X²¹=N or S or R;
X²²=R or S or Y;
X²³=G or A or N or Y;
X²⁴=G or A;
X²⁵=S or A or P;
X²⁶=K or Q;
X²⁷=R or L;
X²⁸=A or C;
X²⁹=T or H;
X³⁰=I or H;
X³¹=S or L;
X³²=R or E;
X³³=D or G;
X³⁴=N or Q or R or D or K;
X³⁵=G or R or W;
X³⁶=Q or A;
X³⁷=S or E;
X³⁸=T or H;
X³⁹=V or S;
X⁴⁰=R or E;
X⁴¹=L or A;
X⁴²=Q or A;
X⁴³=L or A;
X⁴⁴=N or E or S;
X⁴⁵=N or Q;
X⁴⁶=L or P;
X⁴⁷=R or Q;
X⁴⁸=A or G;
X⁴⁹=E or G;
X⁵⁰=D or H;
X⁵¹=T or R or L;
X⁵²=G or P or a deletion;
X⁵³=T or P or a deletion;
X⁵⁴=Y or T or a deletion;
X⁵⁵=Y or S or a deletion;
X⁵⁶=C or A or a deletion;
X⁵⁷=A or P or a deletion;
X⁵⁸=K or a deletion;
X⁵⁹=a carboxy group, a carboxy derivative or one or more amino acids;
and wherein mutual conservative exchanges of any amino acids are admissible as long as the inhibition of the syncytial fusion of trophoblast cells is retained;
wherein the proteins with the sequences VL and VH are interconnected through a linker.

2. The protein according to claim 1, wherein said linker is a peptide chain which consists of at least any five amino acids.

3. The protein according to claim 1 wherein D-amino acids are contained in the sequence.

4. The protein according to claim 1 with the amino acid sequences SEQ ID Nos. 1 to 10.

5. A protein having the sequence SEQ ID No. 11 (consensus sequence).

6. A nucleic acid, especially DNA or RNA, coding for a protein according to claim 1.

7. A plasmid or vector containing a nucleic acid according to claim 6.

8. A medicament containing at least one of the proteins according to claim 1 or at least one nucleic acid coding for the protein.

9. Use of at least one of the proteins according to claim 1 or at least one nucleic acid coding for the protein for preparing a medicament for contraception in mammals.


SEQUENCE LISTING (13 sequences)

SEQ ID NO: 1 - protein, 209 aa, Artificial
  Hybrid sequence
ALTQPSSVSANLGGTVEITCSGGGRSWYGWYQQKSPGSAPVTVIYNNNKRPSDIPSRFSGSKSGSTATLTITGVQADDEAVYYCGSSAGRNAAAFGAGTTLTVLGQSSRSSAVTLDESGGGLQTPGGGLSLVCKASGFDFSNYQMNWIRQAPGKGLEFVAAINSFGNRTGYAAAVKGRATISRDDGQSTVRLQLSNLRAEDTGTYYCAK

SEQ ID NO: 2 - protein, 210 aa, Artificial
  Consensus sequence
ALTQPSSVSANPGGTVKITCSGSSGSYGWYQQKSPGSAPVTVIYNNNKRPSDIPSRFSGSKSGSTATLTITGVQAEDEAVYFCGSRDSSAGYVGIFGAGTTLTVLGQSSRSSSVTLDESGGGLQTPGGALSLVCKASGFSMSSYQMNWIRQAPGKGLEWVAGMNRFANRTNYGSAVKGRATISRDDGQSTVRLQLNNLRAEDTATYYCAK

SEQ ID NO: 3 - protein, 215 aa, Artificial
  Hybrid sequence
ALTQPSSVSANPGGTVKITCSGGSGSWSYGWYQQKSPGSAPVTVIYNNNKRPSDIPSRFSGSKSGSTATLTITGFQADDEAVYYCGNRDWDSSAGYVGIFGAGTTLTVLGXXXXXXAVTLDESGGGLQTPGGGLSLVCKASGFSFSSYQMNWIRQAPGKGLEFVAGISSGFGNRTGYGSAVKGRATISRDNGQSTVRLQLNNLRAEDTGTYYCAK

SEQ ID NO: 4 - protein, 211 aa, Artificial
  Hybrid sequence
ALTQPSSVSANPGETVKITCSGGGSYYGWYQQKAPGSAPVTVIYDNTNRPSNIPSRFSGSKSGSTHTLTITGVQADDEAVYFCGSYDSSAGYVGIFGAGTTLTVLGQSSRSSAVTLDESGGGLQTPGGGLSLVCKASGFTFSDYGMGWVRQAPGKGLEFVARISSGSGSSTGYGSAVKGRATISRDKGQSTVRLQLNNLRAEDTGTYYCAK

SEQ ID NO: 5 - protein, 207 aa, Artificial
  Hybrid sequence
ALTQPSSVSANPGETVKITCSGGGSSYGWYQQKSPGSTPVTVIYNNKNRPSDIPSRFSGSKSGSANTLTITGVQADDEAVYFCGSRADNVGIFGAGTTLTVLGQSSRSSTVTLDESGGGLQTPGRTLSLVCKASGFSFSSYGMAWVRQAPGKGLEFVAGISNTGSSTGYGAAVKGRATISRDNGQSTVRLQLNNLRAEDTGTYYCAK

SEQ ID NO: 6 - protein, 203 aa, Artificial
  Hybrid sequence
ALTQPSSVSANPGETVKLTCSGGSYSSYYGWYQQKSPGSAPVTLIYESNKRPSNIPSRFSGSKSGSTATLTITGVQAEDEAVYYCGGRDSSYVNIFGAGTTLTVLGQSSRSSAVTLDESGGGLQTPGGGLSLVCKASGFSFSSYTMQWVRQAPGKGLEYVAGISSNSRYTYYGSAVKGLCHHLEGQRAEHSEAAAEQPQGGHR

SEQ ID NO: 7 - protein, 211 aa, Artificial; identical to SEQ ID NO: 4
  Hybrid sequence

SEQ ID NO: 8 - protein, 203 aa, Artificial
  Hybrid sequence
ALTQPSSVSANPGGTVKITCSGSSGSYGWYQQKSPGSAPVTVIYNNNKRPSDIPSRFSGSKSGSTATLTITGVQAEDEAVYFCGSRDSSAGYVGIFGAGTTLTVLGQSSRSSSVTLDESGGGLQTPGGALSLVCKASGFSMSSYQMNWIRQAPGKGLEWVAGMNRFANRTNYGSAVKGRCHHLEGRWAEHSEAAAEQPQGGHR

SEQ ID NO: 9 - protein, 210 aa, Artificial
  Hybrid sequence
ALTQPSSVSANLGGTVKITCSGGSSRKYGWYQQKSPGSAPDTVIYSNDNRPSDIPSRFSGSVSGSTNTLTITGVQAEDEAVYFCGSGDWKTGTGMFGAGTTLTVLGQSSRSSAVTLDESGGGLQTPGGTLSLVCKASGFTFSSYAMNWVRQAPGKGLEFVAGIGNTGRYTGYGPAVQGRATISRDNGQSTVRLQAEQPQGGHLPPTSAPK

SEQ ID NO: 10 - protein, 209 aa, Artificial; identical to SEQ ID NO: 1
  Hybrid sequence

SEQ ID NO: 11 - protein, 207 aa, Artificial
  Consensus sequence
ALTQPSSVSANLGGTVEITCSGGSGSYGWFQQKSPGSAPVTVIYVNTNRPSDIPSRFSGSKSGSTATLTITGVQADDEAVYYCGNSAGRNAAAFGAGTTLTVLGQSSRSSAVTLDESGGGLQTPGGGLSLVCKGSGFSFTYQMNWIRQAPGKGLEFVGAISRFGNSTAYGSAVKGRATISRDNGQSTVRLQLNNLRAEDTATYFCAK

SEQ ID NO: 12 - protein, 112 aa, Artificial
  Hybrid sequence
XALTQPSSVSANXGXTVXXTCSGXXXXXXYGWXQQKXPGSXPXTXIYXXXXRPSXIPSRFSGSXSGSXXTLTITGFQAXDEAVYXCGXXXXXXXXXXXXXFGAGTTLTVLGX

SEQ ID NO: 13 - protein, 101 aa, Artificial
  Hybrid sequence
XXVTLDESGGGLQTPGXXLSLVCKXSGFXXXXYXMXWXRQAPGKGLEXXXXXXXXXXXXTXYXXAVXGXXXXXXXXXXXXXXXXXXXXXXXXXXXXXXXXX